(19) United States
(12) Patent Application Publication
(10) Pub. No.: US 2011/0207759 A1
(43) Pub. Date: Aug. 25, 2011

(54) METHOD FOR TREATMENT OF ATHEROSCLEROTIC DISEASE

(76) Inventor: Allen John TAYLOR, Rockville, MD (US)

(21) Appl. No.: 13/030,396

(22) Filed: Feb. 18, 2011

Related U.S. Application Data
(60) Provisional application No. 61/306,068, filed on Feb. 19, 2010.

(51) Int. Cl. A61K 31/505; A61K 31/455; A61P 9/10

(52) U.S. Cl. 514/275; 514/356

(57) ABSTRACT

A method of effecting regression of atherosclerotic disease in a patient whose plasma lipid concentrations are at levels considered normal or optimal. The method involves treating the patient with a combination of a 3-hydroxy-3-methylglutaryl-coenzyme A reductase inhibitor and niacin. Disease regression has been observed over periods as short as 14 months, preferably 8 months.

### CROSS-REFERENCE TO RELATED APPLICATION

The present application is related to and claims priority under 35 U.S.C. §119(e) to U.S. provisional patent application Ser. No. 61/306,068 filed 19 Feb. 2010. This application is incorporated herein in its entirety.

### FIELD OF THE INVENTION

The invention relates to a method of treating a patient having atherosclerotic disease. More particularly, the invention relates to a method of effecting regression of atherosclerotic disease in a patient whose plasma lipid concentrations are at levels considered normal or optimal. The method involves treating the patient with a combination of a 3-hydroxy-3-methylglutaryl-coenzyme A reductase inhibitor and niacin. Disease regression has been observed over periods as short as about 14 months, preferably as short as about 8 months.

### BACKGROUND OF THE INVENTION

The publications and other materials used herein to illuminate the background of the invention, and in particular, cases to provide additional details respecting the practice, are incorporated by reference in their entirety for all that they disclose, and for convenience are referenced in the following text by author and date and are listed alphabetically by author in the appended bibliography.

Therapy with a 3-hydroxy-3-methylglutaryl-coenzyme A reductase inhibitor (sometimes referred to herein as “statin” or “HMG-CoA reductase inhibitor) has been shown to result in reduced plasma levels of low-density lipoprotein cholesterol (“LDL-C”), and thereby provides protection against recurrent coronary heart disease. Niacin therapy also reduces LDL-C plasma concentrations and has been shown to increase levels of high-density lipoprotein (“HDL-C”). It is believed that HDL-C is important in inhibiting atherosclerosis and thus has a cardioprotective effect.

Progressive LDL-C-lowering through statin therapy intensification leads to a 16% odds reduction for cardiovascular mortality and events (Brown et al., 2001). This approach has also been shown to yield improvement in atherosclerosis as measured by carotid intima media thickness (“CIMT”) (Brown et al., 1990) or coronary ultrasonography (Hodis et al., 1998). Because of the residual cardiovascular risk seen with statin monotherapy, treatment with combination therapy, aimed at either further reducing the LDL cholesterol level or at altering levels of HDL-C has been proposed for the treatment of atherosclerotic disease (Taylor et al., 2005). Low levels of HDL-C may be treated as a secondary lipid target through niacin-statin combination therapy. This approach has led to potentially clinically-significant relative risk reductions in clinical coronary event rates within generally small studies (Taylor et al., 2005) and stabilization or regression of atherosclerosis (Brown et al., 2001; Brown et al., 1990; Hodis et al., 1998; Taylor et al., 2005; Taylor et al., 2006; Whitney et al., 2005).

Fixed-dose, extended-release combinations of niacin and simvastatin and similar combinations of niacin and lovastatin are currently marketed under the trademarks Simcor® and Advicor®, respectively. The dominant effect of these medications has been to delay the progression of atherosclerosis (Brown et al., 1990). A clinical trial using the combination of simvastatin (10-20 mg/day) and niacin (2-4 g/day) demonstrated stabilization of coronary atherosclerosis; however, that trial could not demonstrate an additive effect of niacin to the statin (Brown et al., 2001). More recent studies have demonstrated that the addition of niacin to statin monotherapy provides superior effects on atherosclerosis stabilization and regression (Taylor et al., 2004; Taylor et al., 2006).

Guidelines for the treatment of lipid disorders have been revised recently to indicate that an LDL-C plasma level of less than 100 mg/dl is optimal with a call for more intensive LDL-C lowering therapy in certain groups of people. The guidelines also raised the target for HDL-C from 35 mg/dl to 40 mg/dl. See National Cholesterol Educational Program (“NCEP”) Report ATP III at http://www dot nhlbi dot nih dot gov backslash guidelines backslash cholesterol/atp3_rpt.htm. A recent publication reported the design of a randomized clinical trial to address the effects of targeting further LDL-C reduction or initiating HDL-C raising therapy on carotid artery by randomizing patients receiving statin therapy to either ezetimibe or extended-release niacin therapy (Devine et al., 2007). Entry criteria for this study included an LDL-C plasma concentration of less than 100 mg/dl and an HDL-C plasma concentration of less than 50 mg/dl for men and less than 55 mg/dl for women. The primary endpoint for the proposed study was the 14-month between-group change in mean CIMT. At the time the publication, it was unknown and unpredictable whether combination therapy could achieve any decrease in the progression of atherosclerotic disease, much less regression of the disease, in patients already having plasma lipid levels considered optimal.

### SUMMARY OF THE INVENTION

In accordance with the present invention, the present invention provides a method of effecting regression of atherosclerotic disease in a human patient afflicted with such disease which comprises administering to the patient a combination of niacin and 3-hydroxy-3-methylglutaryl-coenzyme A reductase inhibitor in therapeutically effective dosages sufficient to cause regression of such atherosclerotic disease. In one embodiment, the patient is a male patient having an HDL-C plasma concentration equal to or greater than about 40 mg/dl and equal to or less than about 65 mg/dl and an LDL-C plasma concentration equal to or less than about 100 mg/dl. In a second embodiment, the patient is a female patient having an HDL-C plasma concentration equal to or greater than about 45 mg/dl and equal to or less than about 65 mg/dl and an LDL-C plasma concentration equal to or less than about 100 mg/dl. In one embodiment, the 3-hydroxy-3-methylglutaryl-coenzyme A reductase inhibitor is rosuvastatin, lovastatin, atorvastatin, pravastatin, fluvastatin, or simvastatin. In another embodiment, the combination is administered on a substantially daily basis to achieve regression of the atherosclerotic disease over a 14-month period, preferably over an 8-month period. In a further embodiment, the regression of atherosclerotic disease is enhanced in patients taking the niacin for a longer period of time, in patients taking a higher dose of the niacin and in patients taking the niacin on a more regular basis.

### BRIEF DESCRIPTION OF THE DRAWINGS

FIG. 1A-1D show the mean (±standard error) percent changes observed for HDL (FIG. 1A), LDL (FIG. 1B), total cholesterol (FIG. 1C), and triglyceride (FIG. 1D) concentrations over the 14-month study period for 208 subjects randomized to either ezetimibe or niacin that completed the study. P values are given for the comparison between the two treatment groups at 14 months. The vertical bars indicate the standard errors.

FIG. 2 shows the change in mean CIMT (in mm, ±standard error) from the far wall of the bilateral distal common carotid arteries from baseline through 8 and 14 months during treatment with either ezetimibe or niacin when added to statin therapy. The P value is given for the comparison of repeated measures of the CIMT over the 14-month period. The vertical bars indicate the standard errors.

FIG. 3 shows changes in maximum carotid intima-media thickness over the 14-month study period, according to treatment group. The carotid intima-media thickness is the thickness of the far wall of the bilateral distal common carotid arteries, measured in millimeters. The P value is given for the comparison of repeated measures of the carotid intima-media thickness over the 14-month period. The vertical bars indicate standard errors.

FIG. 4 shows bivariate regression plot showing the relationship between change in LDL-C and mean carotid intima-media thickness from baseline to 14 months. Data are shown separately for ezetimibe (upper three lines at a-60 mark) and niacin (lower three lines at −60 mark) for the 208 subjects who completed the 14 month carotid intima-media thickness endpoint. The vertical bars indicate the 95% confidence interval.

FIG. 5 shows the Kaplan-Meier estimates of the incidence of a major cardiovascular event among the 363 study patients, according to treatment group. The P value, calculated with the use of log-rank test, is given for the comparison between the two treatment groups at 14 months. The inset shows the same data on a magnified scale.

FIG. 6 shows the relationship between quartiles of cumulative drug exposure to ezetimibe and niacin and change in CIMT. Cumulative drug exposure was calculated as the product of mean study drug adherence, dose, and time in the study. The relationship between quartiles of drug exposure (lowest, quartile 1, to highest, quartile 4) and change in mean carotid intima-media thickness (CIMT) for all subjects using the method of last observation carried forward is shown. The relationship between quartiles of cumulative drug exposure and change in CIMT is shown separately for ezetimibe (analysis of variance [ANOVA] p_{—}0.05), and niacin (ANOVA p_{—}0.23).

### DETAILED DESCRIPTION OF THE INVENTION

Unless defined otherwise, all technical and scientific terms used herein have the same meaning as is commonly understood by one of skill in the art to which the invention belongs.

It has now been discovered that patients having atherosclerotic disease who have or are at risk of coronary heart disease and whose LDL-C and HDL-C plasma concentrations are at levels that are considered optimal can receive substantial benefit from combination therapy that includes effective doses of a statin and niacin. In particular, regression of the atherosclerotic disease can be achieved over a period as short as about 14 months, preferably about 8 months, with such combination therapy.

Thus, in one aspect, the present invention provides a method of effecting regression of atherosclerotic disease in a human patient afflicted with such disease which comprises administering to the patient a combination of niacin and 3-hydroxy-3-methylglutaryl-coenzyme A reductase inhibitor in therapeutically effective dosages sufficient to cause regression of such atherosclerotic disease. In one embodiment, the patient is a male patient having an HDL-C plasma concentration equal to or greater than about 40 mg/dl and equal to or less than about 65 mg/dl and an LDL-C plasma concentration equal to or less than about 100 mg/dl. In a second embodiment, the patient is a female patient having an HDL-C plasma concentration equal to or greater than about 45 mg/dl and equal to or less than about 65 mg/dl and an LDL-C plasma concentration equal to or less than about 100 mg/dl. In a preferred embodiment, the combination is administered on a substantially daily basis to achieve regression of the atherosclerotic disease over a 14-month period, preferably over an 8-month period. The regression in atherosclerotic disease can be maintained by continued administration of niacin and 3-hydroxy-3-methylglutaryl-coenzyme A reductase inhibitor in therapeutically effective dosages.

The statin employed in the therapeutic method of the invention can be any 3-hydroxy-3-methylglutaryl-coenzyme A reductase inhibitor, including rosuvastatin, lovastatin, atorvastatin, pravastatin, fluvastatin, and simvastatin. In one embodiment, the 3-methylglutaryl-coenzyme A reductase inhibitor is simvastatin. In another embodiment, the 3-methylglutaryl-coenzyme A reductase inhibitor is atorvastatin. The statin is employed at a dosage effective to achieve reduction of LDL-C plasma concentrations. Dosages utilized in statin monotherapy are generally appropriate for the combination therapeutic method of this invention. In one embodiment, dosages for atorvastatin range from about 10 to about 80 mg per day. In another embodiment, simvastatin dosages range from about 20 to about 80 mg per day. In an additional embodiment, therapeutically equivalent dosages of rosuvastatin, lovastatin, pravastatin and fluvastatin are used. Such dosages are well known to the skilled artisan.

Niacin is administered at a dosage of from about 1,000 to about 3,000 mg per day, preferably at a dosage of from about 1,500 to about 2,500 mg per day, most preferably about 2,000 mg per day. The niacin may be administered in an immediate-release form, or, preferably, in a sustained release dosage form that releases the drug substantially uniformly over at least about a 24-hour period. Extended-release niacin tablets are currently available under the trademark, Niaspan®.

The statin and niacin may be administered separately at the dosages described above or as a fixed-dose combination. A fixed-dose combination advantageously contains from about 10 to about 80 mg of the statin, depending on the particular statin employed, and from about 1,000 to about 3,000 mg of niacin, together with conventional pharmaceutical excipients. Proportionally lower concentrations of the active ingredients may be employed if it is desired to administer the fixed-dose combination multiple times per day. The fixed-dose combination may be an immediate-release formulation or an extended-release formulation that releases one or both of the active ingredients substantially uniformly over at least about a 24-hour period. The formulation of such immediate-release or extended release products are well known to those skilled in the field of pharmaceutical science.

The statin and niacin are preferably administered orally. Other routes of administration for one or both of the active ingredients also are contemplated. For example, the medicaments may be administered by intraveneous, subcutaneous or intramuscular injection. Alternatively, transdermal, intranasal, buccal or other routes may be employed. Suitable pharmaceutical practices are described in Remington: The Science and Practice of Pharmacy, 21^{st} Ed., University of Sciences in Philadelphia, Ed., Philadelphia, 2005.

The combination therapy is administered to the patient on a substantially daily basis. Skipping some days, or occasionally reducing the dosage, for example, either through lack of patient compliance with physician's directions or for the purpose of reducing side effects, is considered substantially daily administration.

It has surprisingly been found that the combination therapeutic method of this invention can effect regression of atherosclerotic disease in patients over a relatively short time period. As demonstrated below, disease regression occurs within about 14 months, preferably within about 8 months of therapy. This regression was unexpected, given that the patients' plasma lipid concentrations were at levels generally accepted as optimal.

In addition, it has been surprisingly found that increased cumulative drug exposure as determined by drug adherence, dosage and treatment duration resulted in progressively greater regression of CIMT using the combination of statin and niacin in accordance with the present invention. That is, we discovered a relationship between the longer the patients took the niacin, the more niacin the patients took and the more faithfully (regularly) the patients took the niacin, the better the results in the regression of atherosclerotic disease. This finding was unexpected, given that the patients' plasma lipid concentrations were at levels generally accepted as optimal.

### EXAMPLES

The present invention is described by reference to the following Examples, which are offered by way of illustration and are not intended to limit the invention in any manner. Standard techniques well known in the art or the techniques specifically described below were utilized.

### Example 1

### Methods

Study population: This prospective, randomized, parallel group, open-label, blinded endpoint study was conducted at two centers, Walter Reed Army Medical Center, a university-affiliated, suburban, tertiary care military medical center, and the Washington Adventist Hospital, a private tertiary care hospital located in Takoma Park, Md. The institutional review boards of each facility approved the study, and all volunteers provided written, informed consent. Included subjects were men and women ≧30 years old with either known atherosclerotic coronary or vascular disease (N=279) or a coronary risk equivalent diagnosis including diabetes mellitus (N=38), a 10-year coronary heart disease Framingham risk score ≧20% (N=26), or a coronary calcium score above 200 or 400 (N=20) for women and men respectively. Eligible participants were required to be treated with a stable dose of statin monotherapy with a lipid panel within the past 3 months demonstrating both an LDL-C <100 mg/dL and HDL-C <50 mg/dL (men) or <55 mg/dL (women).

Randomization: Three hundred sixty-three patients were enrolled in the trial. Eligible volunteers were randomly assigned in an allocation-concealed fashion to open-label treatment with either extended-release niacin (Abbott Inc., Abbott Park, Ill.) or ezetimibe 10 mg/d (Merck-Schering Plough Pharmaceuticals, North Wales, Pa.). Niacin was titrated from an initial dose of 500 mg qhs in biweekly increments of 500 mg qhs to the maximum tolerated dose (up to 2000 mg qhs). Randomization was performed using a computer-generated sequence of random numbers. There were no protocol-directed changes in statin medications during the study.

Endpoints: The predefined primary endpoint was the between-group difference in the change in mean CIMT after 14 months. Secondary endpoints included the change in lipid values, a composite endpoint consisting of major adverse cardiovascular events (coronary heart disease death, myocardial infarction, myocardial revascularization, admission to the hospital for an acute coronary syndrome), drug discontinuation due to adverse effects, and health-related quality of life (Schweikert et al., 2008). Clinical cardiovascular endpoints were adjudicated by consensus of two members of the data advisory committee blinded to treatment assignment.

Carotid B-mode Ultrasound: Carotid ultrasonography was performed using a Sonosite Micromax ultrasound system (Bothell, Wash.) equipped with a broadband linear array probe (13 MHz). Participants underwent 3 ultrasound examinations during the study at baseline, 8 and 14 months. Except for the initial 20 baseline examinations performed at the Washington Adventist site, a single sonographer performed all ultrasound examinations. Images were obtained of the far wall of the distal 1 centimeter of the right and left common carotid arteries in the anterior and lateral views. A total of eight images (2 complete sets of 4 separate images) were obtained with standardized ultrasound settings. Baseline CIMT images were used to localize the site of imaging in serial images. Digitized still images from an electrocardiographically-defined diastolic frame were quantitated off-line. A single observer unaware of treatment assignment and patient identification performed measurements of mean and maximal CIMT. Focal atherosclerotic plaque was excluded from the site of CIMT measurement. All measurements were performed in duplicate using an automated border detection algorithm. No scans were excluded due to image quality, and CIMT data were available for all subjects except for one who did not complete the 8-month ultrasound. For mean CIMT, the inter-test variability was 0.0011±0.0125 mm (r=0.997; P<0.001). Intra-observer variability was 0.0001±0.0055 mm (r=0.999; P<0.001). Reader drift was evaluated using a standard image set (10 CIMT images from different patients) which the sonographer serially quantified every 6 months. The maximum difference from any time point between the mean CIMT obtained for these 10 images was ≦0.001 mm.

Cardiovascular risk variables: Following an overnight fast, laboratory measurements included plasma total cholesterol, LDL-C, HDL-C, triglycerides, C-reactive protein, liver-associated enzymes and glucose at baseline, 2, 8 and 14 months. LDL-C was measured using a direct assay. C-reactive protein was measured with a high-sensitivity, commercially available immunoturbidimetric assay, which uses monoclonal antibodies to CRP (Roche Cobas® Integra, Switzerland).

Statistical analysis: The primary endpoint was the change in mean CIMT from baseline to 14 months between the niacin and ezetimibe treatment groups. Based upon a minimum sample size of 150 per group, the trial had an 80% power to detect a difference of CIMT change between agents of 0.02±0.06 mm/year (α=0.05). Clinical and carotid imaging data were held in separate databases for the duration of the trial. Between-group data for continuous variables were assessed with a t-test for independent variables or a Mann-Whitney U test as appropriate. A chi-square test was used for categorical variables. Kaplan-Meier survival analysis was performed using the log rank test. All statistical analyses were performed using SPSS software (version 16, SPSS Inc., Chicago, Ill.). Values are reported as mean±standard deviation or standard error, and median (inter-quartile range) for non-normal distributions. A 2-sided P value of 0.05 was considered statistically significant.

Study design: The study design prespecified the performance of a blinded, interim analysis according to the conservative method of O'Brien and Fleming with an alpha spending function (O'Brien et al. 1979). This was conducted after 180 subjects (60% of the planned sample size) completed the study. An independent data advisory committee evaluated the endpoint data without knowledge of treatment assignment. No formal, a priori stopping boundaries were set for the trial. Based upon efficacy for the primary endpoint, consistency of findings at 8 and 14 months in both mean and maximum CIMT, sensitivity analyses for the statistical stability of the findings, and other secondary analyses showing potentially paradoxical effects of ezetimibe, the committee unanimously recommended that the trial should be terminated. Following termination, final closeout visits were conducted, leaving 208 subjects reported herein with 14 month endpoint data for analysis.

### Example 2

### Results

Baseline characteristics of the subjects who had completed the trial at the time of its termination within the 2 treatment groups were similar (N=208; Table 1). The study population was 80.2% male, 65±11 years old, treated with primarily simvastatin or atorvastatin (90.9%) at a mean dose of 42±25 mg for 6±5 years. Baseline total cholesterol was 147±26 mg/dL, LDL-C 82.1±23.1 mg/dL, HDL-C 42.4±8.5 mg/dL, and TG 134±68 mg/dL. Baseline mean and maximum CIMT were 0.9088±0.1583 mm, and 1.0179±0.1653 mm.

TABLE 1
Baseline Characteristics of 208 Patients Randomly Assigned to
Either Extended Release Niacin or Exetimibe Who Completed the
14 Month CIMT Assessment, According to the Treatment Group *

| | Ezetimibe | | Niacin | | |
|---|---|---|---|---|---|
| | N = 111 | | N = 97 | | P |
| Male gender | 91 | (82) | 76 | (78.4) | 0.51 |
| Age, mean ± SD | 65 ± 11 | | 64 ± 11 | | 0.49 |
| Diabetes mellitus (n, %) | 44 | (39.6) | 31 | (32.0) | 0.25 |
| Hypertension (n, %) | 96 | (86.5) | 82 | (84.5) | 0.69 |
| Tobacco use (n, %) | 5 | (4.5) | 6 | (6.2) | 0.86 |
| Family history of coronary heart disease (n, %) | 42 | (37.8) | 48 | (50.0) | 0.09 |
| History of coronary heart disease (n, %) | | | | | |
| Angina with documented ischemia | 41 | (36.9) | 34 | (35.1) | 0.78 |
| Angiographic coronary disease | 70 | (63.1) | 63 | (64.9) | 0.78 |
| Myocardial infarction | 37 | (34.2) | 27 | (27.8) | 0.32 |
| Percutaneous coronary revascularization | 49 | (44.1) | 29 | (30.9) | 0.05 |
| Coronary bypass surgery | 26 | (23.4) | 25 | (25.8) | 0.69 |
| Medications | | | | | |
| Beta blocker | 83 | (74.8) | 69 | (71.1) | 0.55 |
| Aspirin (baseline and in trial) | 104 | (93.7) | 94 | (96.9) | 0.28 |
| Clopidogrel | 31 | (27.9) | 31 | (32.0) | 0.53 |
| Angiotensin converting enzyme inhibitor | 65 | (58.6) | 61 | (62.9) | 0.52 |
| Statin therapy | | | | | |
| Simvastatin | 43 | (38.7) | 52 | (54.2) | 0.09 |
| Atorvastatin | 63 | (56.8) | 39 | (40.2) | |
| Pravastatin | 2 | (1.8) | 4 | (4.1) | |
| Rosuvastatin | 3 | (2.7) | 2 | (2.1) | |
| Lovastatin | 0 | (0) | 0 | (0) | |
| Mean daily statin dose (mg) | 42 ± 24 | | 42 ± 25 | | 0.98 |
| Duration of statin use (years) | 6.1 ± 5.2 | | 5.2 ± 4.6 | | 0.18 |
| BMI kg/m² ** | 31.0 ± 5.4 | | 30.8 ± 6.7 | | 0.90 |
| Waist circumference (inches) | 41.0 ± 4.8 | | 40.8 ± 5.8 | | 0.81 |
| Systolic blood pressure (mm Hg) | 136 ± 18 | | 132 ± 15 | | 0.11 |
| Diastolic blood pressure (mm Hg) | 75 ± 10 | | 74 ± 10 | | 0.70 |
| Total cholesterol (mg/dL) | 146.6 ± 23.3 | | 145.6 ± 24.0 | | 0.74 |
| LDL (mg/dL) | 83.7 ± 19.9 | | 80.5 ± 17.2 | | 0.22 |
| HDL (mg/dL) | 43.3 ± 8.5 | | 42.5 ± 8.6 | | 0.48 |
| Triglycerides (mg/dL) | 122 | (87-162) | 126 | (94-163) | 0.56 |
| Glucose (mg/dL) | 104.0 ± 27.8 | | 100.1 ± 18.9 | | 0.25 |
| High-sensitivity CRP (mg/L) | 1.9 | (0.8-3.5) | 1.3 | (0.8-4.0) | 0.51 |

* Plus-minus values are means ± SD. To convert values for waist circumference to centimeters, multiply by 2.54. To convert values for cholesterol to millimoles per liter, multiply by 0.02586. To convert values for triglycerides to millimoles per liter, multiply by 0/01129. To convert values for glucose to millimoles per liter, multiply by 0.05551. “CRP” denotes C-reactive protein.
** The body-mass index is the weight in kilograms deivided by the square of the height in meters.

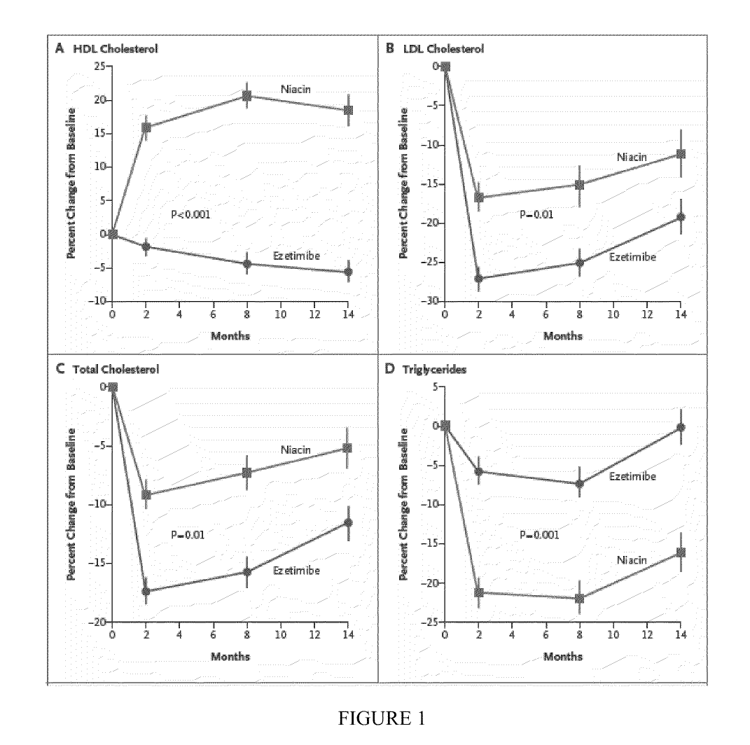

The final change in LDL-C in the ezetimibe group was −17.6±20.1 mg/dL versus −10.0±24.5 mg/dL in the niacin group (P=0.014) (FIG. 1 and Table 2). The final change in HDL-C in the ezetimibe group was −2.8±5.7 mg/dL vs. +7.5±9.2 in the niacin group (P<0.001). Significant reductions in triglycerides were observed in both groups.

TABLE 2
Serial Biomarkers Among 208 Subjects Who
Completed The 14 Month Study According To Treatment Group

| | Baseline | | 2 Months | | 8 Months | | 14 Months | |
|---|---|---|---|---|---|---|---|---|
| | Ezetimibe | Niacin | Ezetimibe | Niacin | Ezetimibe | Niacin | Ezetimibe | Niacin |
| Total | 146.6 ± 23.3 | 143.6 ± 24.0 | 120.2 ± 20.6 | 131.0 ± 22.9 | 122.4 ± 21.5 | 133.6 ± 25.5 | 127.8 ± 22.8 | 136.7 ± 29.3 |
| Cholesterol | | | | | | | | |
| P value | 0.90 | | 0.001 | | <0.001 | | 0.025 | |
| HDL-C | 43.3 ± 8.5 | 42.5 ± 8.6 | 42.2 ± 8.5 | 48.9 ± 10.6 | 41.1 ± 9.0 | 50.7 ± 11.6 | 40.5 ± 7.9 | 49.9 ± 12.2 |
| P value | 0.59 | | <0.001 | | <0.001 | | <0.001 | |
| LDL-C | 83.7 ± 19.9 | 80.5 ± 17.2 | 60.1 ± 16.1 | 66.2 ± 18.1 | 61.6 ± 17.2 | 67.1 ± 19.5 | 66.1 ± 18.8 | 70.5 ± 23.9 |
| P value | 0.34 | | 0.013 | | 0.013 | | 0.12 | |
| Triglycerides | 122 (87-162) | 126 (94-163) | 100 (78-140) | 88 (69-125) | 107 (78-153) | 88 (66-123) | 113 (84-150) | 90 (69-138) |
| P value | 0.56 | | 0.033 | | 0.019 | | 0.018 | |
| Glucose | 104.0 ± 27.8 | 104.1 ± 18.9 | 109.6 ± 39.5 | 109.1 ± 23.9 | 108.2 ± 27.7 | 107.6 ± 28.1 | 110.4 ± 33.4 | 107.4 ± 24.9 |
| P value | 0.20 | | 0.51 | | 0.53 | | 0.34 | |
| C-reactive | 1.9 (0.8-3.6) | 1.3 (0.8-4.0) | 1.2 (0.6-3.0) | 1.1 (0.6-3.1) | 1.3 (0.6-2.9) | 1.1 (0.5-2.4) | 0.6 (0.6-3.1) | 1.0 (0.4-2.9) |
| protein | | | | | | | | |

| | | | | |
|---|---|---|---|---|
| P value | 0.51 | 0.093 | 0.067 | 0.42 |

Values in mg/dL except for C-reactive protein (mg/L).
Data shown as mean ± standard deviation or median (inter-quartile range).

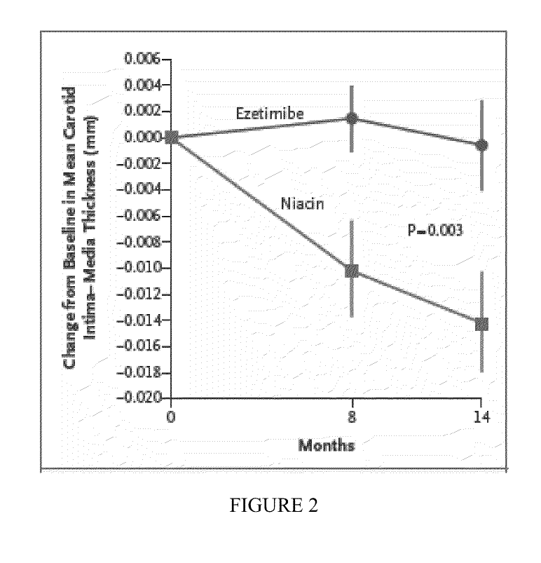

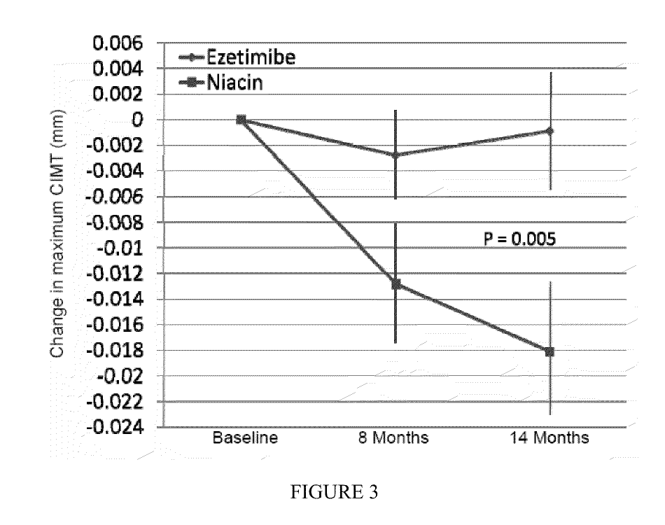

Niacin showed superior efficacy for the change in mean at both 8 and 14 months (FIG. 2) with similar findings for maximal CIMT (Table 3 and FIG. 3). The difference in CIMT change between niacin and ezetimibe from baseline to 14 months was significant (P=0.003). Niacin caused significant reduction in mean and maximal CIMT (regression) at both 8 and 14 months. Significant regression of mean CIMT was observed with niacin between 8 and 14 months (P=0.019). No net change in CIMT was observed with ezetimibe.

TABLE 3
Data on CIMT in the 208 Study Subjects Who Completed
the Study, According to Treatment Group*

| | | |
|---|---|---|
| Ezetimibe (N = 111) | Niacin (N = 97) | P Value |

Baseline

| | | | |
|---|---|---|---|
| Baseline mean CIMT | 0.8957 ± 0.1484 mm | 0.9001 ± 0.1558 mm | 0.83 |
| Baseline max CIMT | 1.0065 ± 0.1548 mm | 1.0092 ± 0.1650 mm | 0.90 |

Change in CIMT- 8 months

| | | | |
|---|---|---|---|
| Mean CIMT | 0.0014 ± 0.0020 mm | −0.0102 ± 0.0030 mm | 0.001 |
| P-Value for Change | 0.48 | 0.001 | |
| From Baseline | | | |
| Max CIMT | −0.0028 ± 0.0031 mm | −0.0128 ± 0.0043 mm | 0.057 |
| P-Value for Change | 0.38 | 0.004 | |
| From Baseline | | | |

Change in CIMT- 14 months

| | | | |
|---|---|---|---|
| Mean CIMT | −0.0007 ± 0.0035 mm | −0.0142 ± 0.0041 mm | 0.012 |
| P-Value for Change | 0.84 | 0.001 | |
| From Baseline | | | |
| Max CIMT | −0.0009 ± 0.0039 mm | −0.0181 ± 0.0050 mm | 0.006 |
| P-Value for Change | 0.81 | <0.001 | |
| From Baseline | | | |

*Plus-minus values are means ± SE. Data are missing for one patient in the niacin group at the 8-month time point.

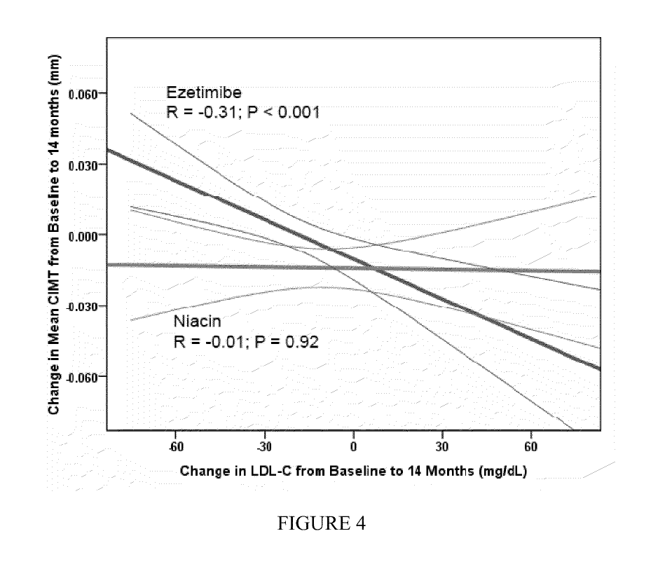

In a post-hoc analysis, the bivariate relationships between changes in LDL-C and mean CIMT were investigated. A significant inverse relationship between LDL-C reductions and CIMT progression was observed in the ezetimibe-treated subjects, such that paradoxical progression of CIMT was seen in the setting of greater reductions in LDL-C(R=−0.31; P<0.001). A similar relationship was not observed with niacin (R=−0.01; P=0.92). See FIG. 4.

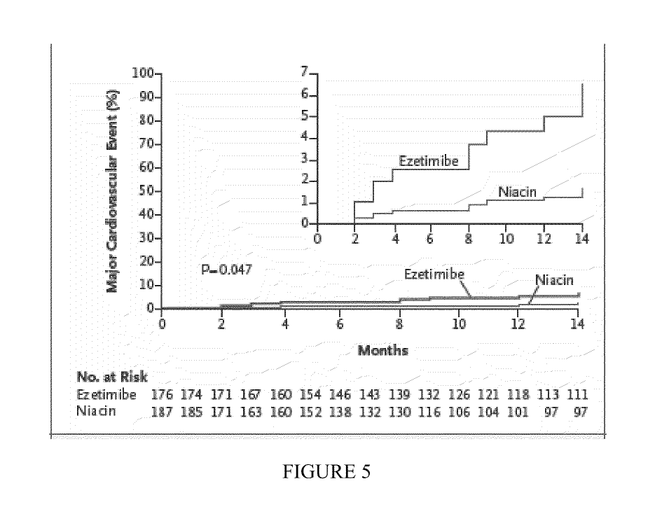

Major adverse cardiovascular events occurred at a significantly higher incidence in the ezetimibe (N=9 subjects; 5.8%) than the niacin group (N=2 subjects; 1.3%; P=0.029) (FIG. 5 and Table 4). The effects of niacin on mean CIMT were consistent across prespecified subgroups including gender, diabetes, quartiles of baseline HDL-C, and median cutpoints for baseline CIMT and C-reactive protein.

TABLE 4
Major Cardiovascular Events Among Subjects Randomized
To Either Ezetimibe Or Niacin In Arbiter 6-Halts

| | Ezetimibe | Niacin | |
|---|---|---|---|
| | N = 165 | N = 160 | P |
| Myocardial infarction | 3 | 1 | |
| Coronary revascularization procedure | 3 | 0 | |
| Cardiovascular death | 5 | 1 | |
| Subjects experiencing a composite | 9 | 2 | 0.04 |
| clinical cardiovascular endpoint* | | | |

*Some subjects may have experienced more than one cardiovascular event. This analysis was performed among subjects who did not withdraw from the study or die from a non-cardiovascular cause.

Among 363 subjects enrolled in the trial, 44 did not complete the study including 16 of 176 in the ezetimibe group (9 withdrawals, 7 deaths; 9.1%) and 28 of 187 (27 withdrawals, 1 death; 14.9%; P=0.09) in the niacin group. There was no significant difference between age, baseline lipids, and baseline CIMT between subjects who withdrew in the 2 treatment groups. Among those who withdrew with laboratory values at 2 months, the LDL response in the ezetimibe and the HDL response in the niacin arm were comparable to those that completed the study. Adverse drug effects were cited as the reason for withdrawal in 3 of 9 ezetimibe and 17 of 27 niacin subjects (P=0.121). Cutaneous flushing was reported in 36.1% of niacin treated subjects.

There was no difference between groups in quality of life at baseline and 14 months. Adherence to study medication by pill counts was 94.9±7.8% with ezetimibe versus 88.2±14.7% with niacin (P<0.001). The final dose of extended-release niacin was 2000 mg/d in 74.7%, 1500 mg/d in 3.2%, 1000 mg/d in 11.6%, and 500 mg/d in 10.5%. There was no significant difference between groups in clinically-directed changes in the statin drug or dose during the study.

This study demonstrated regression of CIMT when extended-release niacin was combined with a statin among patients with CHD or CHD risk equivalents and an LDL-C <100 mg/dL and HDL-C <50-55 mg/dL.

### Example 3

### Further Analysis and Results

After the initial study termination, all actively enrolled patients were contacted and returned for final collection of clinical variables, laboratory data, and blinded CIMT assessment. Among the 363 patients initially enrolled in the trial, 208 patients had completed the entire 14 months of the study period at the time of their final visit (111 ezetimibe, 97 ERN), and 44 had left the study. Final ultrasound examinations could not be obtained in 4 additional subjects after termination of the study (total of 48 participants dropped out), leaving 315 patients for this analysis. In 107 of the 315 patients analyzed, final achieved lipid values and CIMT measurements were performed after study termination at a mean treatment duration of 7±3 months, and these values are included in the primary end point (a last observation carried forward analysis).

The baseline characteristics of the 363 patients enrolled in the trial were similar between the 2 treatment groups (Table 5). A majority of the patients were male (80%), hypertensive (87%), age 65±11 years, and had taken a statin (atorvastatin or simvastatin by 95%) at a mean dose of 42 mg for 6±5 years. There was no difference with respect to age, baseline lipid values, and CIMT between the 48 participants who did not complete the trial and the remainder of the trial participants. In addition, there was no significant difference among baseline covariates, including demographics, blood pressure, lipid levels, baseline therapies, or baseline CIMT between patients who completed the entire 14-month trial (n=208) and patients who completed the trial after study termination (n=107). Baseline and final lipid and biomarker values in the 315 patients who completed the trial are shown in Table 6. Significant reductions in baseline LDL-C and triglycerides were seen with both ezetimibe and ERN. As compared with ERN, patients treated with ezetimibe achieved significantly lower total cholesterol, LDL-C, and HDL-C, and had higher triglyceride values. There was no difference in baseline or final fasting glucose values between the study groups. There was no significant difference between groups in clinically directed changes in the statin drug or dose during the study. Mean study drug adherence rates over the duration of the study exceeded 80% in each arm, and were significantly higher with ezetimibe as compared with ERN (87.5±15.3% vs. 82.1±17.2%, respectively; p=0.005).

TABLE 5
Baseline Characteristics Of 363 Patients Randomly Assigned
To Either Extended Release Niacin Or Exetimibe

| | Ezetimibe | | Niacin | | |
|---|---|---|---|---|---|
| | N = 176 | | N = 187 | | P |
| Male gender | 14491 | (81.8) | 147 | (78.6) | 0.44 |
| Age, mean ± SD | 65 ± 11 | | 65 ± 10 | | 0.63 |
| Diabetes mellitus (n, %) | 72 | (40.9) | 73 | (39.0) | 0.71 |
| Hypertension (n, %) | 154 | (87.5) | 163 | (87.2) | 0.92 |
| Tobacco use (n, %) | 9 | (5.1) | 13 | (7.0) | 0.68 |
| Family history of coronary heart disease (n, %) | 65 | (36.9) | 88 | (47.3) | 0.05 |
| History of coronary heart disease (n, %) | | | | | |
| Angina with documented ischemia | 65 | (36.9) | 58 | (31.2) | 0.25 |
| Angiographic coronary disease | 109 | (61.9) | 116 | (62.0) | 0.98 |
| Myocardial infarction | 59 | (33.5) | 54 | (28.9) | 0.34 |
| Percutaneous coronary revascularization | 70 | (39.8) | 58 | (31.0) | 0.08 |
| Coronary bypass surgery | 44 | (25.0) | 48 | (25.7) | 0.88 |
| Medications | | | | | |
| Beta blocker | 125 | (71.0) | 125 | (67.6) | 0.48 |
| Aspirin (baseline and in trial) | 165 | (93.8) | 176 | (94.1) | 0.88 |
| Clopidogrel | 32 | (24.8) | 30 | (27.0) | 0.70 |
| Angiotensin converting enzyme inhibitor | 100 | (56.8) | 96 | (51.9) | 0.35 |
| Statin therapy | | | | | |
| Simvastatin | 70 | (39.8) | 78 | (41.7) | 0.18 |
| Atorvastatin | 96 | (54.5) | 88 | (47.1) | 0.22 |
| Pravastatin | 5 | (2.8) | 9 | (4.8) | |
| Rosuvastatin | 5 | (2.8) | 8 | (4.3) | |
| Lovastatin | 0 | (0) | 4 | (2.1) | |

| | | | |
|---|---|---|---|
| Mean daily statin dose (mg) | 42 ± 24 | 42 ± 25 | 0.95 |
| Duration of statin use (years) | 6.5 ± 5.6 | 6.1 ± 4.9 | 0.40 |
| BMI kg/m² ** | 30.8 ± 5.6 | 31.3 ± 6.4 | 0.43 |
| Waist circumference (inches) | 40.9 ± 5.0 | 41.6 ± 5.9 | 0.27 |
| Systolic blood pressure (mm Hg) | 137 ± 18 | 134 ± 18 | 0.08 |
| Diastolic blood pressure (mm Hg) | 74 ± 10 | 75 ± 11 | 0.82 |
| CIMT mean thickness, mm | 0.8957 ± 0.1484 | 0.9001 ± 0.1558 | 0.83 |
| CIMT maximal thickness, mm | 1.0065 ± 0.1548 | 1.0092 ± 0.1650 | 0.90 |

Values are mean ± SD or n (%)

TABLE 6
Serum Biomarkers At Baseline And Study Completion

| | Baseline (n = 315) | | Final Values (n = 315) | |
|---|---|---|---|---|
| | Ezetimibe | Niacin | Ezetimibe | Niacin |
| | (n = 161) | (n = 154) | (n = 161) | (n = 154) |
| Total cholesterol, mg/dl | 147.3 ± 28.5 | 147.2 ± 24.8 | 127.5 ± 24.8 | 137.6 ± 30.5 |
| p value, between groups | 0.96 | | 0.002 | |
| p value, change from baseline | | | <0.001 | <0.001 |
| HDL-C, mg/dl | 43.0 ± 8.8 | 42.0 ± 8.3 | 41.1 ± 8.5 | 49.7 ± 11.5 |
| p value, between groups | 0.32 | | <0.001 | |
| p value, change from baseline | | | <0.001 | <0.001 |
| LDL-C, mg/dl | 84.5 ± 23.8 | 83.2 ± 19.4 | 65.5 ± 20.0 | 72.7 ± 25.9 |
| p value, between groups | 0.59 | | 0.007 | |
| p value, change from baseline | | | <0.001 | <0.001 |
| Triglycerides, mg/dl | 118 (87-160) | 129 (89-166) | 111 (82-144) | 91 (69-134) |
| p value, between groups | 0.24 | | 0.012 | |
| p value, change from baseline | | | 0.025 | <0.001 |
| Fasting glucose, mg/dl | 104.0 ± 27.1 | 103.9 ± 29.2 | 111.2 ± 33.8 | 111.5 ± 41.0 |
| p value, between groups | 0.97 | | 0.96 | |
| p value, change from baseline | | | 0.03 | 0.001 |
| hsCRP, mg/l | 1.6 (0.7-3.2) | 1.3 (0.8-3.7) | 1.0 (0.5-2.9) | 1.0 (0.4-2.3) |
| p value, between groups | 0.71 | | 0.21 | |
| p value, change from baseline | | | <0.001 | 0.06 |

Values are mean ± SD.
Median values listed with (interquartile range).
HDLC—high-density lipoprotein cholesterol;
hsCRP = highly-sensitive C-reactive protein;
LDL-C = low-density lipoprotein cholesterol.

Primary end point: The primary end point was assessed among all randomized subjects (n=315) who completed a final CIMT measurement after either 14 months (n=208) or after <14 months of treatment (n=107) among subjects who had not yet completed the study at the time of its termination (Table 7). Treatment with niacin (n=154) resulted in significant reduction (regression) in mean CIMT (−0.0102±0.0026 mm; p<0.001) and maximal CIMT (−0.0124±0.0036 mm; p=0.001) compared to baseline. Treatment with ezetimibe (n=161) had no effect on mean CIMT (−0.0016±0.0024 mm; p=0.088) or maximal CIMT (−0.0005±0.0029 mm; p=0.88) compared to baseline. There was a significant difference between the ezetimibe and niacin treatment groups on the change in CIMT, favoring niacin for both mean CIMT (p=0.016) and maximal CIMT (p=0.01) among the 315 patients completing the trial. Imputing baseline CIMT as the final CIMT (last observation of baseline CIMT carried forward) in the 48 patients who did not complete the trial resulted in no change in the primary outcome.

TABLE 7
Change From Baseline CIMT By Treatment Group For Completing Subjects
And For All Subjects With The Last Observation Carried Forward

| | | | P Value |
|---|---|---|---|
| CIMT | Ezetimibe | Niacin | (Ezetimibe vs. Niacin) |
| Completing subjects | n = 111 | n = 97 | |
| Mean thickness, mm | −0.0007 ± 0.0035 | −0.0142 ± 0.0041 | 0.01 |
| p value, change from baseline | 0.84 | 0.001 | |
| Maximal thickness, mm | −0.0009 ± 0.0039 | −0.0181 ± 0.0050 | 0.006 |
| p value, change from baseline | 0.81 | <0.001 | |
| Last observation carried forward | n = 161 | n = 154 | |
| Mean thickness, mm | −0.0016 ± 0.0024 | −0.0102 ± 0.0026 | 0.016 |
| p value, change from baseline | 0.52 | <0.001 | |
| Maximal thickness, mm | −0.0005 ± 0.0029 | −0.0124 ± 0.0036 | 0.01 |
| p value, change from baseline | 0.88 | 0.001 | |

Data shown as mean ± SE.
CIMT = carotid intima-media thickness.

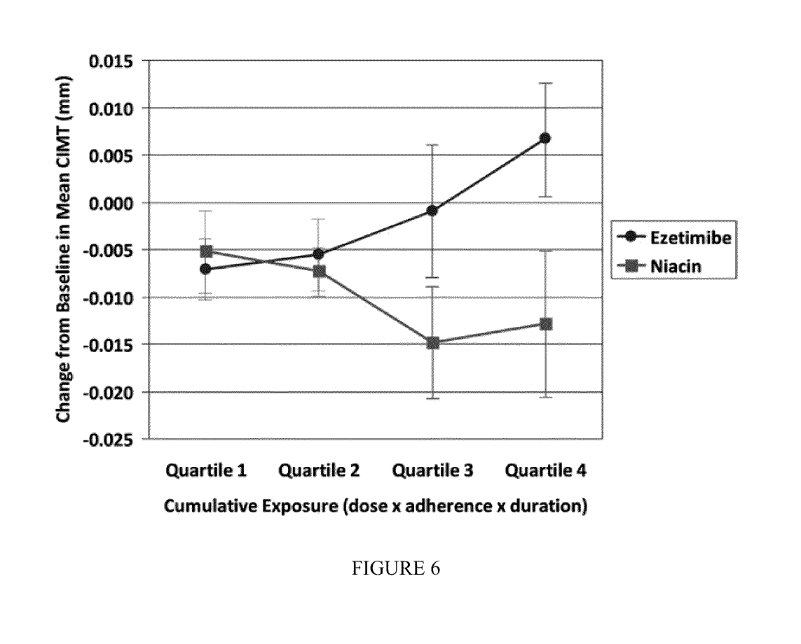

Impact of cumulative drug exposure: Based on differences in study medication adherence, ERN dosage achieved and treatment duration (due to early trial termination) between trial participants, we performed an exploratory analysis to assess the impact of these variables on change in CIMT among niacin and ezetimibe treatment groups. The product of study drug adherence, dosage, and treatment duration was calculated to estimate cumulative exposure to study drug as an integrated measure of drug effect (Holme et al., 2009). The relationships between increasing cumulative drug exposure (lowest, quartile 1, to highest, quartile 4) and change in mean CIMT for all subjects using the method of last observation carried forward are shown in Table 8 and FIG. 6. Increased cumulative drug exposure resulted in regression of CIMT with niacin, and progression of CIMT with ezetimibe. Specifically, comparing participants treated optimally (best-case comparison), defined as those with the highest quartile of study drug adherence and those reaching the target 2,000 mg/day in the niacin group (quartile 4) for the entire 14 months of the study, patients treated with ERN had significant reduction (regression) of mean CIMT from baseline (−0.0128±0.0078 mm), whereas patients treated with ezetimibe experienced CIMT progression (0.0067±0.0059 mm).

TABLE 8
Change From Baseline CIMT Stratified By Quartile Of Increasing
Cumulative Drug Exposure* To Ezetimibe And Niacin

| | | | | | ANOVA |
|---|---|---|---|---|---|
| | Quartile 1 | Quartile 2 | Quartile 3 | Quartile 4 | p Value |
| Ezetimibe | | | | | |
| Adherence, % | 71 ± 22 | 87 ± 8 | 94 ± 1 | 98 ± 1 | <0.001 |
| Maximum does, mg/day | 9 ± 2 | 10 | 10 | 10 | |
| Treatment duration, months | 6.9 ± 2.6 | 13.2 ± 1.4 | 14 | 14 | <0.001 |
| Change in mean CIMT, mm | −0.0071 ± 0.0031 | −0.0051 ± 0.0036 | −0.0009 ± 0.0067 | 0.0067 ± 0.0059 | 0.05 |
| Niacin | | | | | |
| Adherence, % | 63 ± 19 | 80 ± 14 | 87 ± 6 | 98 ± 1 | <0.001 |
| Maximum dose, mg/day | 1271 ± 690 | 1700 ± 441 | 2,000 | 2,000 | <0.001 |
| Treatment duration, months | 8.2 ± 4.1 | 11.0 ± 2.7 | 13.8 ± 0.7 | 14 | <0.001 |
| Change in mean CIMT, mm | −0.0052 ± 0.0041 | −0.0073 ± 0.0027 | −0.0148 ± 0.0060 | −0.0128 ± 0.0078 | 0.23 |
| CIMT p value, ezetimibe v. | 0.71 | 0.63 | 0.13 | 0.048 | |
| niacin within quartile | | | | | |

*Cumulative drug exposure = (adherence × dose × treatment duration). Adherence, dose, and treatment duration expressed as mean ± SD; CIMT expressed as mean ± SE.
ANOVA = analysis of variance; CIMT = carotid intima-media thickness.

Example 2 demonstrated that extended release niacine (ERN) leads to significant regression of CIMT and was superior to ezetimibe when added to chronic statin therapy among 208 high-risk patients who had completed 14 months of randomized, open-label, parallel group treatment in the trial. This comparative-efficacy trial was stopped before all enrolled patients completing the intended 14 months of drug treatment after a planned interim analysis clearly demonstrated the superiority of ERN over ezetimibe in the effect on CIMT. In Example 3, we extend our original observations by examining the effect of the study treatments on CIMT within a last observation carried forward and therapy optimization analysis.

Consistent with the results seen for patients completing 14 months of treatment, among the entire study group (n=315) including 208 subjects treated for the full study duration and 107 subjects treated for a mean of 7 months, therapy with ERN led to significant regression of baseline CIMT among statin-treated patients with an LDL-C <100 mg/dl and an HDL-C <50 or 55 mg/dl. Treatment with ezetimibe did not significantly change CIMT. Comparatively, there was a significant difference between the effect of niacin and ezetimibe on changes in CIMT, favoring niacin. These results strengthen the findings from the interim analysis through the inclusion of patients treated for shorter times (last observation carried forward analysis), which might have been expected to reduce the magnitude of effect between the 2 treatment strategies originally observed. Final results confirm the difference in the primary outcome and preserved magnitude of CIMT regression among patients taking ERN. In addition, the magnitude of effect of ERN on mean CIMT, producing regression (−0.0142±0.0041 mm), over 14 months of treatment in the relatively high risk study population is noteworthy in comparison with other contemporaneous lipid-lowering trials using similar CM/IT methodology.

We have observed that the CIMT response was related to niacin adherence, dose, and increased treatment duration, calculated as cumulative drug exposure. The relationship between cumulative drug exposure and the CIMT effect of ERN supports an expected, direct relationship between increasing intensity of drug exposure (through a composite of dose, adherence, and time) and its effect on atherosclerosis. Final results from this trial confirm the superiority of extended-release niacin over ezetimibe for the end point of change in CIMT and the ability of niacin to induce GMT regression. Increased cumulative drug exposure was related to regression of GMT with niacin, and progression of CIMT with ezetimibe.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### BIBLIOGRAPHY

Brown G., Albers J. J., Fisher L. D. et al. (1990). Regression of coronary artery disease as a result of intensive lipid-lowering therapy in men with high levels of apolipoprotein B [see comments]. N Engl J Med 323(19):1289-1298.
Brown B. G., Zhao X. Q., Chait A. et al. (2001). Simvastatin and niacin, antioxidant vitamins, or the combination for the prevention of coronary disease. N Engl J Med 345(22): 1583-1592.
Devine P. J., Turco M. A., Taylor A. J. (2007). Design and rationale of the ARBITER 6 trial (Arterial Biology for the Investigation of the Treatment Effects of Reducing Cholesterol)-6-HDL and LDL Treatment Strategies in Atherosclerosis (HALTS). Cardiovasc Drugs Ther 21(3):221-225.
Hodis H. N., Mack W. J., LaBree L. et al. (1998). The role of carotid arterial intima-media thickness in predicting clinical coronary events. Ann Intern Med 128(4):262-269.
Holme I, Szarek M, Cater N B, et al. (2009). Adherence-adjusted efficacy with intensive versus standard statin therapy in patients with acute myocardial infarction in the IDEAL study. Eur J Cardiovasc Prey Rehabil 16:315-20.
O'Brien P. C., Fleming T. R. (1979). A multiple testing procedure for clinical trials. Biometrics 35(3):549-556.
Schweikert B., Hahmann H., Leidl R. (2008). Development and first assessment of a questionnaire for health care utilization and costs for cardiac patients. BMC Health Serv Res 2008; 8:187.
Taylor A. J., Sullenberger L. E., Lee H. J., Lee J. K., Grace K. A. (2004). Arterial Biology for the Investigation of the Treatment Effects of Reducing Cholesterol (ARBITER) 2: a double-blind, placebo-controlled study of extended-release niacin on atherosclerosis progression in secondary prevention patients treated with statins. Circulation 110:3512-17.
Taylor A. J., Sullenberger L. E., Lee H. J. (2005). ARBITER 3: Atherosclerosis Regression During Open-label Continuation of Extended-release Niacin following ARBITER 2. Circulation 112[17], II-179. 10-25-2005 (abstract).
Taylor A. J., Lee H. J., Sullenberger L. E. (2006). The effect of 24 months of combination statin and extended-release niacin on carotid intima-media thickness: ARBITER 3. Curr Med Res Opin 22(11):2243-2250.
Whitney E. J., Krasuski R. A., Personius B. E. et al. (2005). A randomized trial of a strategy for increasing high-density lipoprotein cholesterol levels: effects on progression of coronary heart disease and clinical events. Ann Intern Med 142(2):95-104.

1. A method of effecting regression of atherosclerotic disease in a human patient afflicted with such disease which comprises administering to a male patient having an HDL-C plasma concentration equal to or greater than about 40 mg/dl and equal to or less than about 65 mg/dl and an LDL-C plasma concentration equal to or less than about 100 mg/dl or to a female patient having an HDL-C plasma concentration equal to or greater than about 45 mg/dl and equal to or less than about 65 mg/dl and an LDL-C plasma concentration equal to or less than about 100 mg/dl a combination of niacin and 3-hydroxy-3-methylglutaryl-coenzyme A reductase inhibitor in therapeutically effective dosages sufficient to cause regression of such atherosclerotic disease.

2. The method of claim 1, wherein the combination of niacin and 3-hydroxy-3-methylglutaryl-coenzyme A reductase inhibitor is administered to a male or female patient having HDL-C plasma concentration less than or equal to about 50 mg/dl.

3. The method of claim 1, wherein the niacin is administered at a dosage of from about 1,000 to about 3,000 mg per day.

4. The method of claim 1, wherein the niacin is administered at a dosage of from about 1,500 to about 2,500 mg per day.

5. The method of claim 1, wherein the niacin is administered at a dosage of about 2,000 mg per day.

6. The method of claim 4, wherein the niacin is administered as a single controlled release dosage form that releases the drug substantially uniformly over a 24 hour period.

7. The method of claim 1, wherein the 3-hydroxy-3-methylglutaryl-coenzyme A reductase inhibitor is a member selected from the group consisting of rosuvastatin, lovastatin, atorvastatin, pravastatin, fluvastatin, and simvastatin.

8. The method of claim 1, wherein the 3-hydroxy-3-methylglutaryl-coenzyme A reductase inhibitor is simvastatin.

9. The method of claim 1, wherein the 3-hydroxy-3-methylglutaryl-coenzyme A reductase inhibitor is atorvastatin.

10. The method of claim 7, wherein the niacin and the 3-hydroxy-3-methylglutaryl-coenzyme A reductase inhibitor are administered as separate dosage forms over the same time period.

11. The method of claim 7, wherein the niacin and the 3-hydroxy-3-methylglutaryl-coenzyme A reductase inhibitor are administered together as a fixed dose combination.

12. The method of claim 8, wherein the dosage of simvastatin is from about 20 to about 80 mg per day.

13. The method of claim 9, wherein the dosage of atorvastatin is from about 10 to about 80 mg per day.

14. The method of claim 11, wherein the fixed dose combination contains from about 1,000 to about 3,000 mg of niacin and from about 10 mg to about 80 mg of atorvastatin.

15. The method of claim 1, wherein the regression of atherosclerotic disease is determined by measuring diminution of arterial wall build-up.

16. The method of claim 15, wherein the diminution of arterial wall build-up is determined by measuring the carotid intima-media thickness.

17. The method of claim 16, wherein the carotid intima-media thickness is measured by ultrasonography.

18. The method of claim 1, wherein the administration occurs on substantially a daily basis and the regression of atherosclerotic disease occurs over a period of at least 8 months.

19. The method of claim 18, wherein the administration occurs on substantially a daily basis and the regression of atherosclerotic disease occurs over a period of at least 14 months.

20. The method of claim 1, wherein the regression of atherosclerotic disease is enhanced in patients taking the niacin for a longer period of time, in patients taking a higher dose of the niacin and in patients taking the niacin more regularly.
